SEQUENCE LISTING (119 sequences)

SEQ ID NO: 1 - protein, 5 aa, Homo sapiens
IYWQK

SEQ ID NO: 2 - protein, 19 aa, Homo sapiens
ADFPTPSISDFEIPTSNIR

SEQ ID NO: 3 - protein, 10 aa, Homo sapiens
GLYDVVSVLR

SEQ ID NO: 4 - protein, 11 aa, Homo sapiens
ITENPVSTGEK

SEQ ID NO: 5 - protein, 12 aa, Homo sapiens
LLNDEDQVVVNK

SEQ ID NO: 6 - protein, 7 aa, Homo sapiens
ATVGLIR

SEQ ID NO: 7 - protein, 14 aa, Homo sapiens
FYALSASFEPFSNK

SEQ ID NO: 8 - protein, 9 aa, Homo sapiens
GLQTSQDAR

SEQ ID NO: 9 - protein, 12 aa, Homo sapiens
YLAIVHAVFSLR

SEQ ID NO: 10 - protein, 9 aa, Homo sapiens
YSLNSTTWK

SEQ ID NO: 11 - protein, 13 aa, Homo sapiens
LSLSQLNSNPELR

SEQ ID NO: 12 - protein, 11 aa, Homo sapiens
TLLNETPEQIK

SEQ ID NO: 13 - protein, 6 aa, Homo sapiens
LLYIFK

SEQ ID NO: 14 - protein, 17 aa, Homo sapiens
EGPELSPDDPAGLLDLR

SEQ ID NO: 15 - protein, 13 aa, Homo sapiens
VTPEIPAGLPSPR

SEQ ID NO: 16 - protein, 7 aa, Homo sapiens
LEENNHK

SEQ ID NO: 17 - protein, 11 aa, Homo sapiens
SVQYDDVPEYK

SEQ ID NO: 18 - protein, 8 aa, Homo sapiens
YSYNLFSR

SEQ ID NO: 19 - protein, 6 aa, Homo sapiens
ILLWSR

SEQ ID NO: 20 - protein, 16 aa, Homo sapiens
NSTFGSVEVHNLQPEK

SEQ ID NO: 21 - protein, 14 aa, Homo sapiens
EDDQYSHLQGNQLR

SEQ ID NO: 22 - protein, 8 aa, Homo sapiens
WNLGSNAK

SEQ ID NO: 23 - protein, 11 aa, Homo sapiens
DLVVQQAGTNK

SEQ ID NO: 24 - protein, 10 aa, Homo sapiens
TTSVLQWAEK

SEQ ID NO: 25 - protein, 11 aa, Homo sapiens
STVPTDFSSAK

SEQ ID NO: 26 - protein, 8 aa, Homo sapiens
IEVSQLLK

SEQ ID NO: 27 - protein, 21 aa, Homo sapiens
VMYTTQGGGEAWGISVLNPNK

SEQ ID NO: 28 - protein, 24 aa, Homo sapiens
SATLLPSFTVTPTVTESTGTTSHR

SEQ ID NO: 29 - protein, 11 aa, Homo sapiens
LYWQGGPALGR

SEQ ID NO: 30 - protein, 17 aa, Homo sapiens
VLPVGDEVVGIVGYTSK

SEQ ID NO: 31 - protein, 15 aa, Homo sapiens
GPLASQISGLYLPYK

SEQ ID NO: 32 - protein, 11 aa, Homo sapiens
TWNLGETVELK

SEQ ID NO: 33 - protein, 13 aa, Homo sapiens
LGDTFVLTLSDFR

SEQ ID NO: 34 - protein, 13 aa, Homo sapiens
SDLVNEEATGQFR

SEQ ID NO: 35 - protein, 6 aa, Homo sapiens
SFFALR

SEQ ID NO: 36 - protein, 8 aa, Homo sapiens
LASYQAAR

SEQ ID NO: 37 - protein, 9 aa, Homo sapiens
FDPELLFNK

SEQ ID NO: 38 - protein, 10 aa, Homo sapiens
VSQASIDQSR

SEQ ID NO: 39 - protein, 12 aa, Homo sapiens
TSAQHALTSVSR

SEQ ID NO: 40 - protein, 14 aa, Homo sapiens
VSETEYSALEQSTK

SEQ ID NO: 41 - protein, 14 aa, Homo sapiens
YLSITYFTNTPSSR

SEQ ID NO: 42 - protein, 11 aa, Homo sapiens
VLEQLEDLDSR

SEQ ID NO: 43 - protein, 14 aa, Homo sapiens
LPLFPEPLHVFAPR

SEQ ID NO: 44 - protein, 10 aa, Homo sapiens
EFAAALTAER

SEQ ID NO: 45 - protein, 9 aa, Homo sapiens
TEILNQEWK

SEQ ID NO: 46 - protein, 6 aa, Homo sapiens
YYQVPR

SEQ ID NO: 47 - protein, 11 aa, Homo sapiens
GQLVVNPEALK

SEQ ID NO: 48 - protein, 13 aa, Homo sapiens
HPDASVNFSEFSK

SEQ ID NO: 49 - protein, 13 aa, Homo sapiens
GEHPGLSIGDVAK

SEQ ID NO: 50 - protein, 12 aa, Homo sapiens
DPTQPILEALDK

SEQ ID NO: 51 - protein, 13 aa, Homo sapiens
SNSISLDNLKPSR

SEQ ID NO: 52 - protein, 11 aa, Homo sapiens
WSIPLGPIPAR

SEQ ID NO: 53 - protein, 10 aa, Homo sapiens
FPYLEASDYK

SEQ ID NO: 54 - protein, 15 aa, Homo sapiens
LLEQSEWQPTNVDGK

SEQ ID NO: 55 - protein, 18 aa, Homo sapiens
EEPEIDSPGGDIGLSLQR

SEQ ID NO: 56 - protein, 11 aa, Homo sapiens
QLETQADPTGR

SEQ ID NO: 57 - protein, 7 aa, Homo sapiens
LLELLTK

SEQ ID NO: 58 - protein, 10 aa, Homo sapiens
AVPEGFVIPR

SEQ ID NO: 59 - protein, 7 aa, Homo sapiens
LLPPYLR

SEQ ID NO: 60 - protein, 9 aa, Homo sapiens
LVAVGGQPR

SEQ ID NO: 61 - protein, 14 aa, Homo sapiens
LDSGDLLQQAQEIR

SEQ ID NO: 62 - protein, 17 aa, Homo sapiens
SLAQDAPPLPVPGVLLK

SEQ ID NO: 63 - protein, 12 aa, Homo sapiens
EFTVSGNILTIR

SEQ ID NO: 64 - protein, 8 aa, Homo sapiens
GFILTSQK

SEQ ID NO: 65 - protein, 9 aa, Homo sapiens
DEEPLFQLK

SEQ ID NO: 66 - protein, 8 aa, Homo sapiens
LAAFPEDR

SEQ ID NO: 67 - protein, 7 aa, Homo sapiens
SAFVVER

SEQ ID NO: 68 - protein, 10 aa, Homo sapiens
LDQEEAQYQR

SEQ ID NO: 69 - protein, 11 aa, Homo sapiens
ETGQQIGDEVR

SEQ ID NO: 70 - protein, 9 aa, Homo sapiens
AQFADIEPK

SEQ ID NO: 71 - protein, 10 aa, Homo sapiens
TIVSSEVSGK

SEQ ID NO: 72 - protein, 9 aa, Homo sapiens
EIFPQSLPK

SEQ ID NO: 73 - protein, 11 aa, Homo sapiens
SDTIANIQLNK

SEQ ID NO: 74 - protein, 9 aa, Homo sapiens
TGLIEVVLR

SEQ ID NO: 75 - protein, 9 aa, Homo sapiens
GIDIPVLPR

SEQ ID NO: 76 - protein, 7 aa, Homo sapiens
SFLGINK

SEQ ID NO: 77 - protein, 17 aa, Homo sapiens
TIIVTELSPVVAKPQIK

SEQ ID NO: 78 - protein, 21 aa, Homo sapiens
QIVGYAIGTQQATPGPANSGR

SEQ ID NO: 79 - protein, 6 aa, Homo sapiens
SVETIK

SEQ ID NO: 80 - protein, 13 aa, Homo sapiens
LTNYSVTDLNVQR

SEQ ID NO: 81 - protein, 14 aa, Homo sapiens
NVIQLVDVLYNEEK

SEQ ID NO: 82 - protein, 11 aa, Homo sapiens
IDSTEVIYQPR

SEQ ID NO: 83 - protein, 10 aa, Homo sapiens
STGDPQGVIR

SEQ ID NO: 84 - protein, 8 aa, Homo sapiens
YNSDLVQK

SEQ ID NO: 85 - protein, 11 aa, Homo sapiens
GGVEEGPTVLR

SEQ ID NO: 86 - protein, 7 aa, Homo sapiens
TPEEVTR

SEQ ID NO: 87 - protein, 13 aa, Homo sapiens
AGTGGSSGYHYLR

SEQ ID NO: 88 - protein, 12 aa, Homo sapiens
VLNAQELQSETK

SEQ ID NO: 89 - protein, 11 aa, Homo sapiens
EAVPEPVLLSR

SEQ ID NO: 90 - protein, 8 aa, Homo sapiens
GGEIEGFR

SEQ ID NO: 91 - protein, 11 aa, Homo sapiens
AVVFLEPQWYR

SEQ ID NO: 92 - protein, 12 aa, Homo sapiens
YFHHNSDFYIPK

SEQ ID NO: 93 - protein, 16 aa, Homo sapiens
TPIQEEQADAHSTLAK

SEQ ID NO: 94 - protein, 14 aa, Homo sapiens
DLISNINVIVLELK

SEQ ID NO: 95 - protein, 8 aa, Homo sapiens
YTPESLWR

SEQ ID NO: 96 - protein, 7 aa, Homo sapiens
GILIFSR

SEQ ID NO: 97 - protein, 13 aa, Homo sapiens
SLSNYPFDFQGAR

SEQ ID NO: 98 - protein, 11 aa, Homo sapiens
DIQVASNEILR

SEQ ID NO: 99 - protein, 12 aa, Homo sapiens
YGFIEGHVVIPR

SEQ ID NO: 100 - protein, 9 aa, Homo sapiens
FAGVFHVEK

SEQ ID NO: 101 - protein, 17 aa, Homo sapiens
VIPGPPALTLVPAELVR

SEQ ID NO: 102 - protein, 11 aa, Homo sapiens
VVEATAFGLGK

SEQ ID NO: 103 - protein, 12 aa, Homo sapiens
EQEPTQQFIPVK

SEQ ID NO: 104 - protein, 9 aa, Homo sapiens
VSSFVHWIK

SEQ ID NO: 105 - protein, 9 aa, Homo sapiens
LPLALTNWR

SEQ ID NO: 106 - protein, 9 aa, Homo sapiens
ALSQYLTDR

SEQ ID NO: 107 - protein, 12 aa, Homo sapiens
YFWTGLSDIQTK

SEQ ID NO: 108 - protein, 12 aa, Homo sapiens
TGIAGGLWDVLK

SEQ ID NO: 109 - protein, 8 aa, Homo sapiens
SVSNAATR

SEQ ID NO: 110 - protein, 10 aa, Homo sapiens
LSPEYYDLAR

SEQ ID NO: 111 - protein, 12 aa, Homo sapiens
NPQEGLYNELQK

SEQ ID NO: 112 - protein, 14 aa, Homo sapiens
GHDGLYQGLSTATK

SEQ ID NO: 113 - protein, 10 aa, Homo sapiens
AEVAPEEDER

SEQ ID NO: 114 - protein, 9 aa, Homo sapiens
LESVNAELK

SEQ ID NO: 115 - protein, 10 aa, Homo sapiens
AEGLFWQTLR

SEQ ID NO: 116 - protein, 8 aa, Homo sapiens
TLLLSHNR

SEQ ID NO: 117 - protein, 10 aa, Homo sapiens
GILYAAPQLR

SEQ ID NO: 118 - protein, 8 aa, Homo sapiens
SLLSLELK

SEQ ID NO: 119 - protein, 10 aa, Homo sapiens
SPLTTGVYVK

(19) United States
(12) Patent Application Publication
(10) Pub. No.: US 2020/0033359 A1
(43) Pub. Date: Jan. 30, 2020

(54) Immune Profiling Of Tumor Tissue

(71) Applicant: Expression Pathology, Inc., Rockville, MD (US)

(72) Inventors: Todd HEMBROUGH, Gaithersburg, MD (US); Fabiola CECCHI, Washington, DC (US); Sarit SCHWARTZ, Rockville, MD (US); Kerry SCOTT, Germantown, MD (US)

(21) Appl. No.: 16/488,449

(22) PCT Filed: Mar. 2, 2018
(86) PCT No.: PCT/US2018/020693

§ 371 Date: Aug. 23, 2019

Related U.S. Application Data
(60) Provisional application No. 62/467,076, filed on Mar. 3, 2017.

(51) Int. Cl. G01N 33/68; G01N 30/72

(52) U.S. Cl. CPC G01N 33/6848; G01N 2800/60; G01N 2560/00; G01N 30/7233

(57) ABSTRACT

SRM/MRM assays are used to detect and quantitate proteins involved in the process of initiating, inhibiting, maintaining, and/or otherwise modulating a tumor immune response directly in patient tumor tissue. The assays provide an immune profile of the tissue microenvironment, and may be used as part of improved methods of immune-based treatment using agents that manipulate the cancer immune response together with cancer therapeutic agents.

### FIELD OF THE INVENTION

Tumor tissue obtained from a cancer patient is subjected to mass spectrometry-based quantitative proteomic analysis to provide a personalized patient tumor immune profile. Protein assays performed on patient tumor tissue are useful for detecting and quantitatively measuring levels of proteins that: 1) predict optimal immuno-based therapy, 2) associate with a positive response to optimal immuno-based therapy, and/or 3) initiate, inhibit, maintain, promote, and/or otherwise modulate the patient's own immune system to attack the patient's own tumor cells. The cancer patient tumor tissue immune profile provided by the quantitative analysis of these proteins can be used to inform treatment decisions, for example when cancer therapeutic agents designed to manipulate the patient tumor immune response are administered to the patient.

### BACKGROUND

Many cancer drugs act by inhibiting specific proteins that drive tumor cell growth and survival but it often is not possible to know, a priori, which proteins are expressed in a given type of tumor. Accordingly, detecting and quantifying these proteins directly in patient tumor tissue may be used to guide selection of a therapeutic agent, or combination of agents, that will inhibit the protein activity in the tumor and treat the cancer and have a positive effect on overall patient survival. Accurate and precise detection and quantitation of proteins in tumor tissue, including isolated tumor cells present in the tissue, can be performed via mass spectrometry-SRM/MRM analysis of specific peptides derived from proteins expressed in tumor cells extracted from patient tumor tissue by tissue microdissection. The proteins are digested to provide these specific peptides prior to detection. Quantifying groups of proteins in a single SRM/MRM assay of tumor tissue provides for a “profile” or “signature” of protein expression that may be used to inform tumor cell-targeted cancer therapeutics. Examples of quantitative assays to inform treatment decisions with targeted therapeutic agents are IGF-1R protein (see, for example, U.S. Pat. No. 8,728,753) and cMet protein (see, for example, U.S. Pat. No. 9,372,195).

The immune response to a tumor in a cancer patient involves many proteins. These proteins are expressed both normally and aberrantly in many different cell types, such as tumor cells and benign cells in solid tissue and in all the various lineages of lymphocytes. These proteins function collectively to initiate, enhance, modulate, or inhibit a patient's own immune response to his/her own tumor cells. While each protein has a distinct function, the effect on the immune system can depend upon which cell is expressing the protein. In the normal setting, the immune system functions to eradicate tumor cells through a complex molecular signaling process of self vs. non-self recognition mediated through lymphocyte-dependent tumor cell killing. This complex process can be disrupted, however, by tumor cells that evade immune surveillance. A great deal of research has been directed to developing small molecule and biological therapeutic agents to interact with cancer patient tumor immune response proteins to try to manipulate the immune system to attack and kill tumor cells. Successful administration of targeted immunomodulatory therapeutic agents would greatly benefit from a protein expression “profile” or “signature” of the patient immune system landscape in order to determine which target proteins are expressed within the tissue. This immune profile can then inform which therapeutic agent, or combination of agents, will be most likely provide the greatest chance of arming, modulating, manipulating, and/or supporting the patient immune system against tumor cells for optimal patient outcome.

Examples of cancer therapeutic agents designed to manipulate the patient immune system include immune checkpoint inhibitors that target the collection of proteins known as immune checkpoint proteins. The PD-1 protein is an immune checkpoint protein that normally resides on T cells and acts as a type of “off switch” that helps to keep the T cells from attacking other cells in the body. PD-1 acts by binding to PD-L1, a protein present on the surface of some normal (and cancer) cells. When PD-1 binds to PD-L1, it signals the T cell not to attack the cell expressing PD-L1. Some cancer cells express large amounts of PD-L1, which masks them to the immune system and allows them to evade immune surveillance by preventing an attack from T cells. Monoclonal antibodies that target either PD-1 or PD-L1 can “unmask” the cancer cells and boost the immune response against cancer cells. This cancer treatment strategy has shown great promise in treating certain cancers, and examples of PD-1 inhibitors include pembrolizumab (Keytruda) and nivolumab (Opdivo). These drugs have shown to be helpful in treating several types of cancer, including melanoma, non-small cell lung cancer, kidney cancer, head and neck cancers, and Hodgkin's lymphoma. Another example of a PD-L1 inhibitor is atezolizumab (Tecentriq), currently used to treat bladder cancer. These drugs also are being studied for use against other types of cancer. Many other drugs targeting either PD-1 or PD-L1 are currently being tested in clinical trials, either alone or in combination with other drugs.

CTLA4 is another protein on some T cells that acts as an immune checkpoint protein that can have an effect on charging the immune system or keeping the immune system inactive. An example of a drug that binds CTLA4 is ipilimumab (Yervoy), a monoclonal antibody that inhibits CTLA4 and boosts the body's immune response against tumor cells. This drug is currently used to treat melanoma and, like the drugs discussed above, also is being studied against other cancers.

These examples demonstrate how knowledge of the molecular status of both the tumor cells and immune system cells can be used as part of an effective targeted immune-based cancer treatment strategy. The ability to procure homogenous populations of cells directly from patient tumor tissue sections for molecular profiling of histologically-specified cell populations is highly advantageous whereby, for example, tumor cells that may be reside within other types of cells, including normal epithelial cells, endothelial cells, fibroblasts, and immune cells can be studied. Laser Capture Microdissection (LCM) technology (see U.S. Pat. No. 6,867,038) allows compartmentalized molecular analysis of tumor tissue to precisely defined populations of cells. LCM, as well as other commercially available tissue microdissection technologies, including DIRECTOR technology (U.S. Pat. No. 7,381,440), has improved the analysis of tissue samples by allowing the molecular profiling of cells derived from tissue samples to be placed in a pathologically relevant context.

While tissue microdissection provides for purified tumor cell populations, its use is limited when insufficient cells of interest can be procured from the tumor tissue for reliable molecular analysis. Many of the proteins that initiate/maintain the tumor immune response are expressed by small numbers of tumor infiltrating lymphocytes (TILs) and/or immune system cells that may not even be present in the tumor tissue, and therefore analysis of pure populations of cells of interest collected via tissue microdis section may not be possible in some cases. In order to detect the presence of potential immunomodulatory therapeutic candidate proteins in tumor tissue the entire tumor microenvironment may be considered, and not just tumor cells. The methods described below provide the ability to generate a molecular immune profile of patient tumor tissue.

### SUMMARY

SRM/MRM assays are used to detect and quantitate levels of specific proteins in proteomic lysates prepared directly from cancer patient tumor tissue. These proteins are involved in predicting optimal immuno-based therapy, associating with a positive response to optimal immuno-based therapy, and/or initiating/inhibiting/maintaining/modulating a cancer patient's tumor immune response to kill the cancer patient's own tumor cells. The SRM/MRM assays are useful for developing a personalized immune profile of the immune system status of the cancer patient. Once the expression status of these proteins has been determined then specific therapeutic agents can be administered to the patient whereby such agents interact with these immune system proteins to either inhibit or enhance their function to manipulate the cancer patient's own immune system to kill the patient's own tumor cells and thus provide increased patient survival. Such immune system manipulative therapeutic agents comprise biological and/or small molecule agents that can be directly matched to the cancer patient immune system profile, as determined by the presently described SRM/MRM assays, providing for a personalized strategy for immunological cancer treatment.

Methods are provided for determining a protein expression profile in a biological sample of formalin fixed tumor tissue obtained from a cancer patient, by detecting and/or quantifying the level of one or more proteins that function to initiate, maintain, enhance, inhibit, or otherwise modulate the human immune system in a protein digest prepared from the biological sample using mass spectrometry; and calculating the level of the proteins in the biological sample, where the level is a relative level or an absolute level, and where the one or more proteins is selected from the group consisting of B7-1, B7H2, beta-catenin, CALR, CCR4, CD133, CD137, CD137L, CD166, CD28, CD38, CD3G, CD40, CD40L, CD47, CD68, CD70, CD73, CD8A, CEACAM5, cMYC, COX-2, CXCR4, CXCR7, DNMT1, EZH2, GBP2, HMGB1, INFGR2, IL13RA2, IRF1, MyD88, NAMPT, NAPRT1, NYESO1, OX40L, PD-1, STAT3, Beclin-1, PHD2, PI3Kbeta, PI3Kdelta, PI3Kgamma, CEACAM1, IFNγ, STK11, BTK, ARG1, TDO, TGFβ1, CD16, OX40, IL-2, SLFN11, CD39, CD44, CSFIR, GZMB, PRF1, CD206, ONLY, CD3Z, ATF3, CD19, and CTLA

The digest may be fractionated prior to detecting and/or quantifying the amount of the one or more fragment peptides, and the fractionating step may be, for example, liquid chromatography, nano-reverse phase liquid chromatography, high performance liquid chromatography, or reverse phase high performance liquid chromatography. The protein digest of the biological sample may be prepared by the Liquid Tissue protocol. The protein digest may include a protease digest, such as a trypsin digest.

The mass spectrometry method may be, for example, tandem mass spectrometry, ion trap mass spectrometry, triple quadrupole mass spectrometry, hybrid ion trap/quadrupole mass spectrometry, MALDI-TOF mass spectrometry, MALDI mass spectrometry, and/or time of flight mass spectrometry, and the mode of mass spectrometry used may be Selected Reaction Monitoring (SRM), Multiple Reaction Monitoring (MRM), intelligent Selected Reaction Monitoring (iSRM), and/or multiple Selected Reaction Monitoring (mSRM).

In these methods, the fragment peptides may be selected from the group consisting of any or all peptides of SEQ ID NO: 1-11. More specifically, the following peptides may be selected from each of the proteins: B7-1 (SEQ ID NO 1, SEQ ID NO 2), B7H2 (SEQ ID NO 3, SEQ ID NO 4), beta-catenin (SEQ ID NO 5, SEQ ID NO 6), CALR (SEQ ID NO 7, SEQ ID NO 8), CCR4 (SEQ ID NO 9, SEQ ID NO 10), CD133 (SEQ ID NO 11, SEQ ID NO 12), CD137 (SEQ ID NO 13), CD137L (SEQ ID NO 14, SEQ ID NO 15), CD166 (SEQ ID NO 16, SEQ ID NO 17), CD28 (SEQ ID NO 18), CD38 (SEQ ID NO 19, SEQ ID NO 20), CD3G (SEQ ID NO 21, SEQ ID NO 22), CD40 (SEQ ID NO 23), CD40L (SEQ ID NO 24), CD47 (SEQ ID NO 25, SEQ ID NO 26), CD68 (SEQ ID NO 27, SEQ ID NO 28), CD70 (SEQ ID NO 29), CD73 (SEQ ID NO 30, SEQ ID NO 31), CD8A (SEQ ID NO 32, SEQ ID NO 33), CEACAM5 (SEQ ID NO 34), cMYC (SEQ ID NO 35, SEQ ID NO 36), COX-2 (SEQ ID NO 37, SEQ ID NO 38), CXCR4 (SEQ ID NO 39), CXCR7 (SEQ ID NO 40, SEQ ID NO 41), DNMT1 (SEQ ID NO 42, SEQ ID NO 43), EZH2 (SEQ ID NO 44, SEQ ID NO 45), GBP2 (SEQ ID NO 46, SEQ ID NO 47), HMGB1 (SEQ ID NO 48, SEQ ID NO 49), INFGR2 (SEQ ID NO 50, SEQ ID NO 51), IL13RA2 (SEQ ID NO 52, SEQ ID NO 53), IRF1 (SEQ ID NO 54, SEQ ID NO 55), MyD88 (SEQ ID NO 56, SEQ ID NO 57), NAMPT (SEQ ID NO 58, SEQ ID NO 59), NAPRT1 (SEQ ID NO 60, SEQ ID NO 61), NYESO1 (SEQ ID NO 62, SEQ ID NO 63), OX40L (SEQ ID NO 64, SEQ ID NO 65), PD-1 (SEQ ID NO 66), STATS (SEQ ID NO 67), Beclin-1 (SEQ ID NO 68), PHD2 (SEQ ID NO 69, SEQ ID NO 70), PI3Kbeta (SEQ ID NO 71, SEQ ID NO 72), PI3Kdelta (SEQ ID NO 73, SEQ ID NO 74), PI3Kgamma (SEQ ID NO 75, SEQ ID NO 76), CEACAM1 (SEQ ID NO 77, SEQ ID NO 78), IFNγ (SEQ ID NO 79, SEQ ID NO 80), STK11 (SEQ ID NO 81, SEQ ID NO 82), BTK (SEQ ID NO 83, SEQ ID NO 84), ARG1 (SEQ ID NO 85, SEQ ID NO 86), TDO (SEQ ID NO 87, SEQ ID NO 88), TGFβ1 (SEQ ID NO 89, SEQ ID NO 90), CD16 (SEQ ID NO 91, SEQ ID NO 92), OX40 (SEQ ID NO 93), IL-2 (SEQ ID NO 94), SLFN11 (SEQ ID NO 95, SEQ ID NO 96), CD39 (SEQ ID NO 97, SEQ ID NO 98), CD44 (SEQ ID NO 99, SEQ ID NO 100), CSF1R (SEQ ID NO 101, SEQ ID NO 102), GZMB (SEQ ID NO 103, SEQ ID NO 104), PRF1 (SEQ ID NO 105, SEQ ID NO 106), CD206 (SEQ ID NO 107, SEQ ID NO 108), ONLY (SEQ ID NO 109, SEQ ID NO 110), CD3Z (SEQ ID NO 111, SEQ ID NO 112), ATF3 (SEQ ID NO 113, SEQ ID NO 114) TLR8 (SEQ ID NO 115, SEQ ID NO 116), CD19 (SEQ ID NO 117, SEQ ID NO 118), and CTLA4 (SEQ ID NO 119).

In the methods described above, the tissue may be paraffin embedded tissue. The tissue may be obtained from a tumor, such as a primary tumor or secondary tumor.

Advantageously, at least one fragment peptide is quantified. Quantifying the fragment peptide maybe achieved by, for example, comparing an amount of the fragment peptide in one biological sample to the amount of the same fragment peptide in a different and separate biological sample. In another quantification method, the amount of the fragment peptide in a biological sample is determined by comparison to an added internal standard peptide of known amount having the same amino acid sequence. The internal standard peptide may be an isotopically labeled peptide, such as a peptide containing one or more heavy stable isotopes selected from ¹⁸O, ¹⁷O, ³⁴S, ¹⁵N, ¹³C, ²H and combinations thereof.

Detecting and/or quantifying the amount of at least one fragment peptide in the protein digest may be used to indicate the presence of the corresponding protein and an association with cancer in the subject. The results of detecting and/or quantifying the amount of the at least one fragment peptide, or the level of the corresponding protein may be correlated to the activation status of the immune system of a cancer patient. This correlating step may be combined with detecting and/or quantifying the amount of other proteins or peptides from other proteins to provide additional information about the molecular status of the tumor cells of the cancer patient.

Any of these methods may be combined with administering to a patient or subject from which the biological sample was obtained a therapeutically effective amount of a cancer therapeutic agent, where the cancer therapeutic agent and/or amount of the cancer therapeutic agent administered is based upon detection of and/or amount of any one or more (in multiplex) fragment peptides of SEQ ID NO 1-119 from the list of proteins whereby the cancer therapeutic agent is an immunomodulatory cancer therapeutic agent that interacts with one or more of the proteins to initiate, enhance, manipulate, and/or otherwise modulate the cancer patient immune response to attack and kill the patient tumor cells. Detection of and/or amount of any one or more (in multiplex) fragment peptides of SEQ ID NO 1-119 from the list of proteins can be used to predict the therapeutic outcome for a patient treated with one or more of the immunomodulatory cancer therapeutic agents that interacts with one or more of the proteins to initiate, enhance, manipulate, and/or otherwise modulate the cancer patient immune response to attack and kill the patient tumor cells.

In these methods, detection of and/or quantification of any one or more (in multiplex) fragment peptides of SEQ ID NO 1-119 from the list of proteins may be used to detect a positive immune system activation status whereby the patient immune system is actively detecting, attacking, and killing patient's own tumor cells.

These methods may be combined with analysis of other oncoproteins that drive growth of the patient tumor cells, where a targeted cancer therapeutic agent that inhibits or modulates the function of the oncoprotein to inhibit growth of the patient tumor cells is administered to the patient simultaneously and in combination with an immunomodulatory cancer therapeutic agent that interacts with one or more of the proteins to initiate, enhance, manipulate, and/or otherwise modulate the cancer patient immune response to attack and kill the patient tumor cells.

### DETAILED DESCRIPTION

Methods and compositions are provided for specific mass spectrometry-SRM/MRM assays that may be used to develop an immune profile for a cancer patient. Specific protease-digested peptides from immunomodulatory proteins are detected and precisely quantitated in proteomic lysates prepared directly from patient tumor tissue. The process and assays can be used to analyze the immune landscape of a patient's tumor and to guide improved methods of treatment with optimal cancer therapeutic agents that induce and support an active and successful immune response to the patient's own tumor cells. Specifically, the methods include: obtaining a biological sample from a cancer patient such as, for example, formalin fixed paraffin embedded tumor tissue; collecting cells from the tumor tissue, optionally using tissue microdissection; preparing a lysate for mass spectrometry analysis from the collected cells (using, for example, the Liquid Tissue reagents and protocol described in U.S. Pat. No. 7,473,532); analyzing the lysate using SRM/MRM assays, where the assays may performed individually or in multiplex; and utilizing protein detection/quantitation data from the SRM/MRM assays to develop the immune profile. These methods generate SRM/MRM assay data that can be used to determine improved treatment methods for a patient in which therapeutic agents are selected that initiate, modulate, effect, enhance, and otherwise manipulate the patient's immune system by directly interacting with one or more of the proteins detected and/or quantitated by the SRM/MRM assays.

Determining a patient immune profile by the described SRM/MRM assays may be performed on a variety of patient-derived samples including but not limited to blood, urine, sputum, pleural effusion, inflammatory fluid surrounding a tumor, normal tissue, and/or tumor tissue. While all of these types of patient-derived biological samples can be analyzed, advantageously the sample is formalin fixed paraffin-embedded (“FFPE”) patient tumor tissue.

Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde (commonly termed 10% neutral buffered formalin), followed by embedding the fixed whole tissue in paraffin wax for long term storage at room temperature. Molecular analytical methods that can analyze formalin fixed cancer tissue are likely to be the most accepted and heavily utilized methods for analysis of cancer patient tissue.

The most widely-used conventional methodology used to study protein expression in cancer patient tissue, and especially in FFPE tissue, is immunohistochemistry (IHC). IHC methodology uses antibody-based detection. The results of an IHC test are most often interpreted by a pathologist or histotechnologist, and this interpretation is subjective and cannot provide quantitative data that may be predictive of sensitivity to therapeutic agents that target specific proteins. In addition, studies involving IHC assays, such as the Her2 IHC test, suggest the results obtained from such tests may be wrong or misleading. This is likely because different laboratories use different rules for classifying positive and negative IHC status. Each pathologist running a test also may use different criteria to decide whether the results are positive or negative. In most cases, this happens when the test results are borderline, i.e. the results are neither strongly positive nor strongly negative. In other cases, cells present in one area of the cancer tissue section can test positive while cells in a different area of the cancer tissue section can test negative.

Inaccurate IHC test results may mean that patients diagnosed with cancer do not receive the best possible care. If all or a specific region/cells of tumor tissue is truly positive for a specific protein but test results classify it as negative, physicians are unlikely to administer the correct therapeutic treatment to the patient. If tumor tissue is truly negative for expression of a specified protein but test results classify it as positive, physicians may use a specific therapeutic treatment even though the patient not only is unlikely to receive any benefit but also is exposed to the agent's secondary risks. Accordingly, there is great clinical value in the ability to precisely detect and correctly evaluate quantitative levels of specific immune-based proteins in tumor tissue so that the patient will have the greatest chance of receiving a successful immunomodulatory treatment regimen while at the same time reducing unnecessary toxicity and other side effects.

Precise detection and correct evaluation of quantitative levels of specific immune-based proteins in tumor tissue are very effectively determined in a mass spectrometer by SRM/MRM methodology. This methodology detects and quantifies unique fragment peptides from specific proteins, including immune-based proteins, in which the SRM/MRM signature chromatographic peak area of each peptide is determined within a complex peptide mixture present in, for example, a Liquid Tissue lysate (see U.S. Pat. No. 7,473,532). The methods described in U.S. Pat. No. 7,473,532 may conveniently be carried out using the Liquid Tissue reagents and protocol available from Expression Pathology Inc. (Rockville, Md.). Quantitative levels of proteins are determined by the SRM/MRM methodology whereby the SRM/MRM signature chromatographic peak area of an individual specified peptide from each protein in a biological sample is compared to the SRM/MRM signature chromatographic peak area of a known amount of a “spiked” internal standard for each of the individual fragment peptides.

In one embodiment, the “spiked” internal standard is a synthetic version of the same exact protein-derived fragment peptide where the synthetic peptide contains one or more amino acid residues labeled with one or more heavy isotopes, such as ²H, ¹⁸O, ¹⁷O, ¹⁵N, ¹³C, or combinations thereof. Such isotope labeled internal standards are synthesized so that mass spectrometry analysis generates a predictable and consistent SRM/MRM signature chromatographic peak that is different and distinct from the native fragment peptide chromatographic signature peak and which can be used as comparator peak. Accordingly, when the internal standard is “spiked” in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original proteomic preparation from the biological sample. Quantitative data for fragment peptides are displayed according to the amount of proteomic lysate analyzed per sample.

In order to develop and perform the SRM/MRM assay for a fragment peptide for a given protein, additional information beyond simply the peptide sequence can be utilized by the mass spectrometer. This additional information is used to direct and instruct the mass spectrometer (e.g., a triple quadrupole mass spectrometer) to perform the correct and focused analysis of a specific fragment peptide. The additional information about a target peptide may include one or more of: the mono isotopic mass of each peptide; its precursor charge state; the precursor m/z value; the m/z transition ions; and the ion type of each transition ion. This additional information provides the mass spectrometer with the correct directives to allow analysis of a single isolated target peptide within a very complex protein lysate. An SRM/MRM assay may be effectively performed on a triple quadrupole mass spectrometer or an ion trap/quadrupole hybrid instrument. These types of a mass spectrometers presently are considered to be the most suitable instruments for analyzing a single isolated target peptide within a very complex protein lysate that may consist of hundreds of thousands to millions of individual peptides from all the proteins contained within a cell. SRM/MRM assays can, however, be developed and performed on other types of mass spectrometer, including MALDI, ion trap, ion trap/quadrupole hybrid, or triple quadrupole instruments.

The foundation for a single SRM/MRM assay to detect and quantitate a specific protein in a biological sample is identification and analysis of one or more fragment peptides derived from the larger, full length version of the protein. This is because mass spectrometers are highly efficient, proficient, and reproducible instruments when analyzing very small molecules such as a single fragment peptide while mass spectrometers cannot efficiently, proficiently, or reproducibly detect and quantitate full length, intact proteins. One molecule of peptide derives from one molecule of protein and therefore measurement of the molar amount of a fragment peptide gives a direct measurement of the molar amount of the corresponding protein.

A candidate peptide for which a single SRM/MRM assay for an individual protein can be developed is, theoretically, each and every individual peptide generated by complete protease digestion of intact full length proteins as, for example, digestion with trypsin. However, the most advantageous peptide to assay by SRM/MRM for a given protein, and the specifically-defined assay characteristics about each peptide, are not predictable a priori, and must be empirically discovered and determined. It also is found that for some proteins no peptide has presently been identified that can be used to detect and reproducibly quantitate the corresponding protein.

This lack of predictability is especially true when identifying the best SRM/MRM peptide for analysis in a protein lysate such as a Liquid Tissue lysate from formalin fixed paraffin embedded tissue. The presently described SRM/MRM assays designate one or more protease-digested peptides (tryptic digested peptides) for each protein whereby each peptide has been determined to be an advantageous peptide for SRM/MRM assay in Liquid Tissue lysates prepared from formalin fixed patient tissue. Each of the identified peptides can be reproducibly detected and quantitated.

The presently described SRM/MRM assays detect and quantitate proteins that can be used to develop an immune profile of the patient tumor tissue microenvironment. This collection of proteins provide a variety of functions that initiate, inhibit, maintain, modulate, and associate with/predict optimal, or at least preferred, therapeutic agents to induce a successful personalized tumor immune response. These proteins include, but are not limited to, growth factors, growth factor receptors, extracellular matrix proteins, nuclear transcription factors, epithelial cell differentiation factors, immune cell differentiation factors, cell/cell recognition factors, self vs. tumor recognition factors, immune cell activation factors, immune cell inhibiting factors, and immune checkpoint proteins. Each of these individual proteins within this collection of proteins can be expressed by a wide variety of cells in a cancer patient including but not limited to all varieties of solid tissue cells such as epithelial tumor cells, normal epithelial cells, normal fibroblasts, tumor-associated fibroblasts, normal endothelial cells, tumor-associated endothelial cells, normal mesenchymal cells, and tumor-associated mesenchymal cells. Each of these proteins also can be expressed by a wide variety of blood-born white blood cells including but not limited to all varieties of lymphocytes such as B cells, T cells, macrophages, dendrites, mast cells, natural killer cells, eosinophils, neutrophils, and basophils. In many cases each of these individual proteins can be expressed by both solid tissue cells and blood-born tissue cells.

The cellular expression patterns of each of these proteins can be very different depending on the health status of the individual. Under normal and usual healthy conditions these proteins maintain a healthy immune system whereby cellular recognition of self is balanced with recognition of non-self, primarily through the major histocompatibility complex (MHC). The MHC is a set of cell surface proteins essential for the immune system to recognize foreign molecules in vertebrates, which in turn determines histocompatibility.

The main function of MHC molecules is to bind to peptide fragments derived from pathogens and display them on the cell surface for recognition by the appropriate T-cells so an immune response can be mounted against the pathogen. MHC molecules mediate interactions of white blood cells which are immune cells, with other white blood cells or with solid tissue body cells. The MHC determines compatibility of donors for organ transplant, as well as one's susceptibility to an autoimmune disease via cross-reacting immunization. In humans, the MHC is also called the human leukocyte antigen (HLA). In a cell, protein molecules of the host's own phenotype or of other biologic entities are continually synthesized and degraded. Each MHC molecule on the cell surface displays a molecular fraction of a protein, called an epitope. The presented antigen can be either self or non-self. If the antigen is recognized as self then an organism's immune system is prevented from targeting its own cells with an immune killing response.

The MHC not only protects the body from pathogens but also plays a major role in the natural control of cancer cells. Cancer cells contain many mutated proteins and aberrantly expressed proteins that may be displayed by the MHC to alert the immune system. Tumor cells may also express normal proteins but in unusual places, unusual ways, and/or in abnormal amounts providing signals to either mobilize or inhibit an immune response. A normal cell will display peptides from normal cellular protein turnover on its class I MHC and immune system cells (white blood cells) will not be activated in response to them due to central and peripheral tolerance mechanisms that are mediated by specific proteins expressed normally on the surface of the white blood cells. When a cell expresses foreign proteins, such as after viral infection or in the case of aberrant protein expression by a cancer cell, a fraction of the class I MHC will display these peptides on the cell surface. Consequently, those white blood cells specific for the MHC:peptide complex will recognize and kill those cells, including cancer cells presenting the aberrant peptide. Alternatively, class I MHC itself can serve as an inhibitory ligand for that population of white blood cells, called natural killer cells (NKs), that kill virus-infected cells and cancer cells. Reduction in the normal levels of surface class I MHC, a mechanism employed by some viruses during immune evasion or in certain tumors, will help tumor cells to evade immune-mediated killing by inactivating NK-mediated cell killing.

Cancer cells can evade immune-mediated killing on other ways. An example of cancer cells utilizing another strategy to evade immune surveillance is in the case of cancer cells aberrantly expressing the PD-L1 checkpoint protein. Programmed death-ligand 1 (PD-L1) is a transmembrane protein that plays a major role in suppressing the immune system during particular events such as pregnancy, tissue allografts, autoimmune disease and other disease states such as cancer. Normally the immune system reacts to foreign antigens where there is some accumulation in the lymph nodes or spleen which triggers a proliferation of antigen-specific CD8+ T cells that express PD-1. The binding of PD-L1 to PD-1 transmits an inhibitory signal which reduces the proliferation of these CD8+ T cells thereby signaling the immune system to disregard the cancer cells.

Upregulated aberrant expression of PD-L1 by cancer cells allows the cancer cells to evade the host immune system. An analysis of 196 tumor specimens from patients with renal cell carcinoma found that high tumor expression of PD-L1 was associated with increased tumor aggressiveness and a 4.5-fold increased risk of death. Ovarian cancer patients with higher expression of PD-L1 show a significantly poorer prognosis than those with lower expression. It is also known that PD-L1 expression correlates inversely with intraepithelial CD8+ T-lymphocyte count, suggesting that PD-L1 on tumor cells may suppress antitumor CD8+ T cells. Many PD-L1 inhibitors are either in routine cancer therapy use now or are in development as immuno-based cancer therapeutic agents and these agents show good response rates in many patients.

Immuno-based cancer therapy strategies are designed to initiate, modulate, strengthen or manipulate a patient's own immune system to fight and kill the patient's own cancer cells. Many forms of immunotherapy are becoming powerful new approaches for the treatment of cancer. The SRM/MRM assay methods described herein provide the ability to provide quantitative protein expression data such as, for example, the PD-L1/PD-1 immune checkpoint proteins in patient tumor cells, to inform potential treatment strategies with immune-based cancer therapeutic agents in order to initiate and/or modulate the balance of the tumor-activated immune response. An example of a single SRM/MRM assay for an immune checkpoint protein is described for the PD-L1 protein in PCT/US2015/010386.

The presently described SRM/MRM assays detect and quantitate expression of unique proteins expressed by many different cell types, where each protein is involved in initiating and/or modulating the immune system reaction to cancer cells. Each of the assays describes at least one optimal peptide that was identified to detect and measure a single protein whereby each assay can be performed individually or in multiplex with other peptides for other proteins. The protein and peptide listing is shown in Table 1. The proteins for which these assays have been developed are listed in Table 1 by one or more common and/or alternative names:
B7-1 (cluster of differentiation 80, CD80),
B7H2 (cluster of differentiation 275, CD275),
beta-catenin,
CALR (calreticulin, calregulin),
CCR4 (cluster of differentiation 194, CD194),
CD133 (cluster of differentiation 133).
CD137 (cluster of differentiation 137, TNFRSF9),
CD137L (cluster of differentiation 137 ligand),
CD166 (cluster of differentiation 166, CD6L, MEMD),
CD28 (cluster of differentiation 28),
CD38 (cluster of differentiation 38, cyclic ADP ribose hydrolase),
CD3G (cluster of differentiation 3G),
CD40 (duster of differentiation 40),
CD40L (cluster of differentiation 40 ligand. CD154),
CD47 (duster of differentiation 47, integrin associated protein),
CD68 (cluster of differentiation 68),
CD70 (cluster of differentiation 70,
CD73 (cluster of differentiation 73, ecto-5′-nucleotidase),
CD8A (cluster of differentiation 8A).
CEACAM5 (carcinoembryonic antigen-related cell adhesion molecule 5, CD66E),
cMYC (v-myc avian myelocytomatosis viral oncogene homolog),
COX-2 (cyclooxygenase 2),
CXCR4 (cluster of differentiation 184,
CXCR7 (C-X-C chemokine receptor type 7, GPR159),
DNMT1 (DNA cytosine-5-methyltransferase 1),
EZH2 (enhancer of zeste homolog 2),
GBP2 (interferon-induced guanylate-binding protein 2),
HMGB1 (high-mobility group protein 1),
IFNGR2 (interferon gamma receptor 2),
IL13RA2 (interleukin-13 receptor subunit alpha-2),
IRF1 (interferon regulatory factor 1),
MyD88 (myeloid differentiation primary response gene 88),
NAMPT (nicotinamide phosphoribosyltransferase),
NAPRT1 (nicotinate phosphoribosyl-transferase),
NYESO1 (New York Esophageal Squamous Cell Carcinoma 1),
OX40L (cluster of differentiation 252).
PD-1 (programmed cell death protein-1),
STAT3 (signal transducer and activator of transcription 3),
Beclin-1 (BECN1),
PHD2 (prolyl hydroxylase domain-containing protein 2),
PI3Kbeta (phosphatidylinositol-4, 5-bisphosphate 3-kinase-beta),
PI3Kdelta (phosphatidylinositol-4, 5-bisphosphate 3-kinase-delta),
PI3Kgamma (phosphatidylinositol-4, 5-bisphosphate 3-kinase-gamma),
CEACAM1 (carcinoembryonic antigen-related cell adhesion molecule 1, Cluster of Differentiation 66a, CD66a),
IFNγ (interferon type II),
STK11 (liver kinase B1, LKB1),
BTK (Bruton's tyrosine kinase),
ARG1 (arginase),
TDO (tryptophan 2, 3-dioxygenase),
TGFβ1 (transforming growth factor beta 1),
CD16 (cluster of differentiation 16, FCGR3A),
OX40 (CD134, tumor necrosis factor receptor superfamily member 4),
IL-2 (interleukin 2),
SLFN11 (Schlafen family member 11),
CD39 (Ectonucleoside triphosphate diphosphohydrolase-1),
CD44 (phagocytic glycoprotein-1),
CSFIR (colony stimulating factor 1 receptor),
GZMB (granzyme B),
PRF1 (Perforin-1),
CD206 (mannose receptor).
GNLY (Granulysin),
CD3Z (T-cell receptor T3 zeta chain),
ATF3 (Cyclic AMP-dependent transcription factor),
TLR8 (Toll-like receptor 8),
CD19 (B-lymphocyte antigen CD19), and
CTLA4 (cytotoxic T-lymphocyte-associated protein 4).

Surprisingly, it was found that many potential peptide sequences from the proteins listed in Table 1 are unsuitable or ineffective for use in mass spectrometry-based SRM/MRM assays for reasons that are not immediately evident. As it was not possible to predict the most suitable peptides for MRM/SRM assay, it was necessary to experimentally identify, is possible, modified and unmodified peptides in actual Liquid Tissue lysates to develop a reliable and accurate SRM/MRM assay for each designated protein. While not wishing to be bound by any theory, it is believed that some peptides might, for example, be difficult to detect by mass spectrometry because they do not ionize well or produce fragments distinct from other proteins. Peptides may also fail to resolve well in chromatography separation or may adhere to glass or plastic ware.

The peptides found in Table I were derived from their respective designated proteins by protease digestion of all the proteins within a complex Liquid Tissue lysate prepared from cells procured from formalin fixed cancer tissue. Unless noted otherwise, in each instance the protease was trypsin. The Liquid Tissue lysate was then analyzed by mass spectrometry to determine those peptides derived from a designated protein that are detected and analyzed by mass spectrometry. Identification of a specific preferred subset of peptides for mass spectrometric analysis is based on discovery under experimental conditions of which peptide or peptides from a protein ionize in mass spectrometry analyses of Liquid Tissue lysates, and thus demonstrate the ability of the peptide to result from the protocol and experimental conditions used in preparing a Liquid Tissue lysate to be analyzed by the methodology of mass spectrometry.

The optimal peptides for this collection of proteins were discovered as follows. Protein lysates from cells procured directly from formalin (formaldehyde) fixed tissue were prepared using the Liquid Tissue reagents and protocol that comprises collecting cells into a sample tube via tissue microdissection followed by heating the cells in the Liquid Tissue buffer for an extended period of time. Once the formalin-induced cross linking was negatively affected, the tissue/cells were then digested to completion in a predictable manner using a protease, as for example including, but not limited to, the protease trypsin. Each protein lysate was turned into a collection of peptides by digestion of intact polypeptides with the protease. Each Liquid Tissue lysate was analyzed (e.g., by ion trap mass spectrometry) to perform multiple global proteomic surveys of the peptides where the data was presented as identification of as many peptides as could be identified by mass spectrometry from all cellular proteins present in each protein lysate. An ion trap mass spectrometer or another form of a mass spectrometer capable of performing global profiling for identification of as many peptides as possible from a single complex protein/peptide lysate is typically employed. Ion trap mass spectrometers however may be the best type of mass spectrometer for conducting global profiling of peptides.

Once as many peptides as possible were identified in a single MS analysis of a single lysate under the conditions employed, then that list of peptides was collated and used to determine the proteins that were detected in that lysate. That process was repeated for multiple Liquid Tissue lysates, and the very large list of peptides was collated into a single dataset. That type of dataset can be considered as representing the peptides that can be detected by mass spectrometry in the type of biological sample that was analyzed (after protease digestion), and specifically in a Liquid Tissue lysate of the biological sample, and thus includes the peptides for each of the designated proteins.

In one embodiment, the tryptic peptides identified as useful in the determination of absolute or relative amounts of the designated proteins are listed in Table 1. Each of these peptides was detected by mass spectrometry in Liquid Tissue lysates prepared from formalin fixed, paraffin embedded tissue. Thus, each peptide can be used to develop a quantitative SRM/MRM assay for a designated protein in human biological samples, including directly in formalin fixed patient tissue.

Specific and unique characteristics about specific fragment peptides from each designated protein were developed by analysis of all fragment peptides on both an ion trap and triple quadrupole mass spectrometers. That information must be determined experimentally for each and every candidate SRM/MRM peptide directly in Liquid Tissue lysates from formalin fixed samples/tissue; because, interestingly, not all peptides from any designated protein can be detected in such lysates using SRM/MRM as described herein, indicating that fragment peptides not detected cannot be considered candidate peptides for developing an SRM/MRM assay for use in quantitating peptides/proteins directly in Liquid Tissue lysates from formalin fixed samples/tissue.

Specific transition ion characteristics for a given peptide may be used to not only detect a particular fragment peptide but to quantitatively measure this fragment peptide in formalin fixed biological samples. These data indicate absolute amounts of this fragment peptide as a function of the molar amount of the peptide per microgram of protein lysate analyzed. Assessment of corresponding protein levels in tissues based on analysis of formalin fixed patient-derived tissue can provide diagnostic, prognostic, and therapeutically-relevant information about each particular patient.

In one embodiment, methods are provided for measuring the level of each of the proteins listed in Table I in a biological sample, comprising detecting and/or quantifying the amount of one or more fragment peptides in a protein digest prepared from the biological sample using mass spectrometry; and calculating the level of modified or unmodified protein in said sample; where the level is a relative level or an absolute level. In a related embodiment, quantifying one or more modified or unmodified fragment peptides comprises determining the amount of each of the fragment peptides in a biological sample by comparison to an added internal standard peptide of known amount, where each of the fragment peptides in the biological sample is compared to an internal standard peptide having the same amino acid sequence. In some embodiments the internal standard is an isotopically labeled internal standard peptide comprising one or more heavy stable isotopes selected from ¹⁸O, ¹⁷O, ³⁴S, ¹⁵N, ¹³C, ²H or combinations thereof. Because one molecule of a given peptide is derived from a single molecule of a protein, the molar amount of the peptide is a direct measure of the molar amount of the protein present.

The method for measuring the level of a designated protein a biological sample described herein (or fragment peptides as surrogates thereof) may be used as a diagnostic indicator of cancer in a patient or subject. In one embodiment, the results from measurements of the level of a designated protein may be employed to determine the diagnostic stage/grade/status of a cancer by correlating (e.g., comparing) the level of the protein found in a tissue with the level of that protein found in normal and/or cancerous or precancerous tissues. In another embodiment, the results from measurements of the level of a designated protein may be employed to determine which cancer therapeutic agents to treat a cancer patient with and thus the most optimal cancer treatment regimen.

The tissue immune landscape is highly complex whereby multiple proteins expressed by multiple types of solid tissue cells and localized/non-localized immune cells require multiple assays for multiple therapeutic agent indications. This level of protein assay complication can be analyzed by the presently described SRM/MRM assays. These assays are designed to simultaneously detect and quantitate many different proteins with a variety of molecular functions including but not limited to soluble proteins, membrane-bound proteins, nuclear factors, differentiation factors, proteins that modulate cell-to-cell interactions, secreted proteins, immune checkpoint proteins, immune inhibitory proteins, cytokines, and lymphocyte-activating/inhibiting factors.

Tissue microdissection is advantageously utilized to procure pure populations of tumor cells from patient tumor tissue for protein expression analysis using the described SRM/MRM assays in order to determine the immune profile that specifically defines tumor cell status for the patient. Tissue microdissection of tumor tissue may be performed using the process of laser induced forward transfer of cells and cell populations utilizing DIRECTOR technology. The method describing the use of a DIRECTOR slide for laser induced forward transfer of tissue via utilization of an energy transfer interlayer coating is described in U.S. Pat. No. 7,381,440, the contents of which are hereby incorporated by reference in their entirety. However, microdissecting pure populations of tumor cells may likely ignore the protein expression signature/profile of the cells expected to kill the tumor cells, i.e. tumor infiltrating lymphocytes (TILS). This limitation can be overcome by utilizing tissue microdis section to procure, in addition to a pure population of tumor cells, a pure population of TILs whereby areas of the tissue containing large populations of TILs can be collected and processed for protein expression analysis using the described SRM/MRM assays. Through collection and analysis of two distinct cell populations, the patient immune profile can be determined to inform the preferred or optimal treatment regimen for the patient whereby the immune system can be modulated by specific immune-modulating agents for optimal immune-mediated tumor cell killing and the tumor cells can be targeted by targeted therapeutic agents and/or immune-mediated tumor cell killing.

While tissue microdissection produces pure populations of specified cell populations from patient tissue for SRM/MRM analysis, the majority of tumor tissues do not show suitably large areas of distinct populations of TILs to be microdissected. In most cases, TILs are sparsely interspersed amongst the heterogeneous complex tissue microenvironment so that relatively pure populations of tumor cells can be effectively analyzed but analysis of pure populations of TILs is not routinely effective. Tumor tissue-derived TILs express many proteins important to informing the positive manipulation of the immune response using immune system modulatory agents and thus should be analyzed. This limitation can be overcome by preparing an analyzable protein lysate for the described SRM/MRM assays from the entire area of the tumor microenvironment present within the patient tissue. This lysate contains a proteomic representation of the entire complex milieu of many different cell types including, but not limited to, tumor cells, benign non-tumor cells, and immune cells. In this way a highly complex patient-specific immune profile can be determined, capturing the entire immune landscape of the patient tumor environment. In addition, analysis of purified populations of tumor cells as collected by tissue microdis section of a serial section from the same tissue can be compared and contrasted to the overall tumor microenvironment landscape. This approach functionally separates the tumor cell profile from the immune cell profile to identify immune response proteins most likely expressed by localized TILs and/or immune cells not present in the tissue sample, and the effect those proteins may have on the tumor immune landscape.

The presently described SRM/MRM assays detect and quantitate expression of specific proteins in lysates prepared from solid tumor tissue; however, unless pure populations of cells are collected and analyzed these assays cannot provide detailed information about which cells express which proteins. This may be important because aberrant protein expression is common in the tumor microenvironment, as for example when tumor cells express immune inhibitory factors that are usually expressed solely by normal cells, normal lymphocytic cells, and/or TILs. Thus, when expression of candidate therapeutic protein targets has been detected and quantitated by the described SRM/MRM assays a follow-up assay may be necessary to provide the missing cellular localization information. The method to achieve cellular expression context is immunohistochemistry. Understanding which proteins are expressed within the tumor microenvironment and which cells express these proteins may advantageously inform optimal treatment decisions to modulate the patient's own immune response to seek out and kill the tumor cells. The presently described SRM/MRM assays and analysis process provide the ability to detect and quantitate protein targets of immunomodulatory cancer therapeutic agents directly in patient tumor tissue.

An advantageous approach for tumor cell killing is to use a combination therapy where immunomodulatory agents are used in combination with tumor cell targeting agents synergistically for optimal patient response. SRM/MRM assays can be used to determine the quantitative expression status in patient tumor tissue of oncoprotein targets for which inhibitory therapeutic agents have been developed. Examples of SRM/MRM assays to determine the quantitative status of oncoproteins are described for the Met protein (see U.S. Pat. No. 9,372,195) and the IGF-1R protein (see U.S. Pat. No. 8,728,753). The drugs crizotinib and cabozantinib inhibit Met protein function while figitumumab and cixutumumab inhibit IGF-1R protein function. The information from these assays can be combined with information from the presently described SRM/MRM assays to understand the immune status of the tumor tissue. Together, both datasets can be used to inform a treatment regimen for a targeted and immune-based combinatorial therapeutic approach. Thus, for example, if a patient's tumor cells are determined by the SRM/MRM methodology to overexpress both the PD-L1 protein and the Met protein then a logical combinatorial treatment regimen might include administering nivolumab (PD-1 inhibitor) or atezolizumab (PD-L1 inhibitor) in combination with crizotinib (Met inhibitor). The result of such an approach would be to optimally utilize therapeutic agents that specifically target and kill the tumor cells along with arming the patient immune system to attack and kill the tumor cells.

Because both nucleic acids and protein can be analyzed from the same Liquid Tissue biomolecular preparation it is possible to generate additional information about drug treatment decisions from the nucleic acids in the same sample analyzed with the presently described SRM/MRM assays. A specific protein can be found by the presently described SRM/MRM assays to be expressed by certain cells at increased levels while at the same time information about the mutation status of specific genes and/or the nucleic acids and proteins they encode (e.g., mRNA molecules and their expression levels or splice variations) can be obtained. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

TABLE 1

| Protein | SEQ ID NO | Peptide Sequence |
|---|---|---|
| B7-1 | 1 | IYWQK |
| | 2 | ADFPTPSISDFEIPTSNIR |
| B7H2 | 3 | GLYDVVSVLR |
| | 4 | ITENPVSTGEK |
| Beta-catenin | 5 | LLNDEDQVVVNK |
| | 6 | ATVGLIR |
| CALR | 7 | FYALSASFEPFSNK |
| | 8 | GLQTSQDAR |
| CCR4 | 9 | YLAIVHAVFSLR |
| | 10 | YSLNSTTWK |
| CD133 | 11 | LSLSQLNSNPELR |
| | 12 | TLLNETPEQIK |
| CD137 | 13 | LLYIFK |
| CD137L | 14 | EGPELSPDDPAGLLDLR |
| | 15 | VTPEIPAGLPSPR |
| CD166 | 16 | LEENNHK |
| | 17 | SVQYDDVPEYK |
| CD28 | 18 | YSYNLFSR |
| CD38 | 19 | ILLWSR |
| | 20 | NSTFGSVEVHNLQPEK |
| CD3G | 21 | EDDQYSHLQGNQLR |
| | 22 | WNLGSNAK |
| CD40 | 23 | DLVVQQAGTNK |
| CD40L | 24 | TTSVLQWAEK |
| CD47 | 25 | STVPTDFSSAK |
| | 26 | IEVSQLLK |
| CD68 | 27 | VMYTTQGGGEAWGISVLNPNK |
| | 28 | SATLLPSFTVTPTVTESTGTTSHR |
| CD70 | 29 | LYWQGGPALGR |
| CD73 | 30 | VLPVGDEVVGIVGYTSK |
| | 31 | GPLASQISGLYLPYK |
| CD8A | 32 | TWNLGETVELK |
| | 33 | LGDTFVLTLSDFR |
| CEACAM5 | 34 | SDLVNEEATGQFR |
| cMYC | 35 | SFFALR |
| | 36 | LASYQAAR |
| COX-2 | 37 | FDPELLFNK |
| | 38 | VSQASIDQSR |
| CXCR4 | 39 | TSAQHALTSVSR |
| CXCR7 | 40 | VSETEYSALEQSTK |
| | 41 | YLSITYFTNTPSSR |
| DNMT1 | 42 | VLEQLEDLDSR |
| | 43 | LPLFPEPLHVFAPR |
| EZH2 | 44 | EFAAALTAER |
| | 45 | TEILNQEWK |
| GBP2 | 46 | YYQVPR |
| | 47 | GQLVVNPEALK |
| HMGB1 | 48 | HPDASVNFSEFSK |
| | 49 | GEHPGLSIGDVAK |
| INFGR2 | 50 | DPTQPILEALDK |
| | 51 | SNSISLDNLKPSR |
| IL13RA2 | 52 | WSIPLGPIPAR |
| | 53 | FPYLEASDYK |
| IRF1 | 54 | LLEQSEWQPTNVDGK |
| | 55 | EEPEIDSPGGDIGLSLQR |
| MyD88 | 56 | QLETQADPTGR |
| | 57 | LLELLTK |
| NAMPT | 58 | AVPEGFVIPR |
| | 59 | LLPPYLR |
| NAPRT1 | 60 | LVAVGGQPR |
| | 61 | LDSGDLLQQAQEIR |
| NYESO1 | 62 | SLAQDAPPLPVPGVLLK |
| | 63 | EFTVSGNILTIR |
| OX40L | 64 | GFILTSQK |
| | 65 | DEEPLFQLK |
| PD-1 | 66 | LAAFPEDR |
| STAT3 | 67 | SAFVVER |
| Beclin-1 | 68 | LDQEEAQYQR |
| PHD2 | 69 | ETGQQIGDEVR |
| | 70 | AQFADIEPK |
| PI3Kbeta | 71 | TIVSSEVSGK |
| | 72 | EIFPQSLPK |
| PI3Kdelta | 73 | SDTIANIQLNK |
| | 74 | TGLIEVVLR |
| PI3Kgamma | 75 | GIDIPVLPR |
| | 76 | SFLGINK |
| CEACAM1 | 77 | TIIVTELSPVVAKPQIK |
| | 78 | QIVGYAIGTQQATPGPANSGR |
| IFNY | 79 | SVETIK |
| | 80 | LTNYSVTDLNVQR |
| STK11 | 81 | NVIQLVDVLYNEEK |
| | 82 | IDSTEVIYQPR |
| BTK | 83 | STGDPQGVIR |
| | 84 | YNSDLVQK |
| ARG1 | 85 | GGVEEGPTVLR |
| | 86 | TPEEVTR |
| TDO | 87 | AGTGGSSGYHYLR |
| | 88 | VLNAQELQSETK |
| TGFβ1 | 89 | EAVPEPVLLSR |
| | 90 | GGEIEGFR |
| CD16 | 91 | AVVFLEPQWYR |
| | 92 | YFHHNSDFYIPK |
| OX40 | 93 | TPIQEEQADAHSTLAK |
| IL-2 | 94 | DLISNINVIVLELK |
| SLFN11 | 95 | YTPESLWR |
| | 96 | GILIFSR |
| CD39 | 97 | SLSNYPFDFQGAR |
| | 98 | DIQVASNEILR |
| CD44 | 99 | YGFIEGHVVIPR |
| | 100 | FAGVFHVEK |
| CSF1R | 101 | VIPGPPALTLVPAELVR |
| | 102 | VVEATAFGLGK |
| GZMB | 103 | EQEPTQQFIPVK |
| | 104 | VSSFVHWIK |
| PRF1 | 105 | LPLALTNWR |
| | 106 | ALSQYLTDR |
| CD206 | 107 | YFWTGLSDIQTK |
| | 108 | TGIAGGLWDVLK |
| GNLY | 109 | SVSNAATR |
| | 110 | LSPEYYDLAR |
| CD3Z | 111 | NPQEGLYNELQK |
| | 112 | GHDGLYQGLSTATK |
| ATF3 | 113 | AEVAPEEDER |
| | 114 | LESVNAELK |
| TLR8 | 115 | AEGLFWQTLR |
| | 116 | TLLLSHNR |
| CD19 | 117 | GILYAAPQLR |
| | 118 | SLLSLE |
| CRLA4 | 119 | SPLTTGVYVK |

1. A method of determining a protein expression profile in a biological sample of formalin fixed tumor tissue obtained from a cancer patient, the method comprising
detecting and quantifying an amount of one or more fragment peptides in a protein digest prepared from said biological sample using mass spectrometry; and
calculating the amount of one or more proteins corresponding to the one or more fragment peptides in said biological sample;
and
wherein said one or more proteins are selected from the group consisting of B7-1, B7H2, beta-catenin, CALR, CCR4, CD133, CD137, CD137L, CD166, CD28, CD38, CD3G, CD40, CD40L, CD47, CD68, CD70, CD73, CD8A, CEACAM5, cMYC, COX-2, CXCR4, CXCR7, DNMT1, EZH2, GBP2, HMGB1, INFGR2, IL13RA2, IRF1, MyD88, NAMPT, NAPRT1, NYESO1, OX40L, PD-1, STAT3, Beclin-1, PHD2, PI3Kbeta, PI3Kdelta, PI3Kgamma, CEACAM1, IFNγ, STK11, BTK, ARG1, TDO, TGFβ1, CD16, OX40, IL-2, SLFN11, CD39, CD44, CSFIR, GZMB, PRF1, CD206, GNLY, CD3Z, ATF3, CD19, and CTLA4.

2. The method of claim 1, further comprising the step of fractionating said protein digest prior to detecting and/or quantifying the amount of said one or more fragment peptides.

3. The method of claim 2, wherein said fractionating step is selected from the group comprising gel electrophoresis, liquid chromatography, capillary electrophoresis, nano-reverse phase liquid chromatography, high performance liquid chromatography and reverse phase high performance liquid chromatography.

4. (canceled)

5. The method of claim 1, wherein said protein digest comprises a protease digest.

6. The method of claim 5, wherein said protein digest comprises a trypsin digest.

7. The method of claim 1, wherein said mass spectrometry comprises tandem mass spectrometry, ion trap mass spectrometry, triple quadrupole mass spectrometry, hybrid ion trap/quadrupole mass spectrometry, MALDI-TOF mass spectrometry, MALDI mass spectrometry, and/or time of flight mass spectrometry.

8. The method of claim 7, wherein a mode of mass spectrometry used is Selected Reaction Monitoring (SRM), Multiple Reaction Monitoring (MRM), intelligent Selected Reaction Monitoring (iSRM), and/or multiple Selected Reaction Monitoring (mSRM).

9. The method of claim 8, wherein said one or more fragment peptides are selected from the group consisting of peptides having the sequences of SEQ ID NO: 1-119.

10. The method of claim 1, wherein the tissue is paraffin embedded tissue.

11. (canceled)

12. The method of claim 1, wherein quantifying said one or more fragment peptides comprises comparing the amount of said one or more fragment peptides in the biological sample to an amount of the same fragment peptide in a different and separate biological sample.

13. The method of claim 1, wherein quantifying said one or more fragment peptides comprises determining the amount of said one or more fragment peptides in the biological sample by comparison to an added internal standard peptide of known amount having the same amino acid sequence.

14. The method of claim 13, wherein the internal standard peptide is an isotopically labeled peptide.

15. The method of claim 14, wherein the isotopically labeled internal standard peptide comprises one or more heavy stable isotopes selected from ¹⁸O, ¹⁷O, ³⁴S, ¹⁵N, ¹³C, ²H and a combination thereof.

16. The method of claim 1, wherein detecting and quantifying the amount of the one or more fragment peptides in the protein digest indicates the presence of the corresponding protein and an association with cancer in the patient.

17. The method of claim 1, further comprising correlating results of said detecting and quantifying the amount of said one ore more fragment peptides, or a level of the corresponding protein, to the activation status of the immune system of the patient.

18. The method of claim 17, wherein correlating the results of detecting and/or quantifying the amount of said one or more fragment peptides or the level of said corresponding protein to the activation status of the immune system of the patient is combined with detecting and/or quantifying an amount of other proteins or peptides from other proteins.

19. The method of claim 1, further comprising administering to the patient from which said biological sample was obtained a therapeutically effective amount of a cancer therapeutic agent, wherein the cancer therapeutic agent and/or amount of the cancer therapeutic agent administered is based upon detection of and/or amount of any of the one or more fragment peptides, wherein the one or more fragment peptides are selected from peptides having the sequences of SEQ ID NO 1-119.

20. The method of claim 19, further comprising administering to said patient an immunomodulatory cancer therapeutic agent that interacts with the one or more proteins.

21. A method of determining immune system activation status of a patient, the method comprising
detecting and quantifying an amount of one or more fragment peptides in a protein digest prepared from a biological sample of formalin fixed tumor tissue obtained from the patient using mass spectrometry; and
calculating the amount of one or more proteins corresponding to the one or more fragment peptides in said biological sample;
wherein said one or more proteins are selected from the group consisting of B7-1, B7H2, beta-catenin, CALR, CCR4, CD133, CD137, CD137L, CD166, CD28, CD38, CD3G, CD40, CD40L, CD47, CD68, CD70, CD73, CD8A, CEACAM5, cMYC, COX-2, CXCR4, CXCR7, DNMT1, EZH2, GBP2, HMGB1, INFGR2, IL13RA2, IRF1, MyD88, NAMPT, NAPRT1, NYESO1, OX40L, PD-1, STAT3, Beclin-1, PHD2, PI3Kbeta, PI3Kdelta, PI3Kgamma, CEACAM1, IFNγ, STK11, BTK, ARG1, TDO, TGFβ1, CD16, OX40, IL-2, SLFN11, CD39 CD44, CSF1R, GZMB, PRF1, CD206 ONLY, CD3Z, ATF3, TLR8, CD19, and CTLA4.

22. The method of claim 21, wherein said one or more fragment peptides are selected from the group consisting of peptides having the sequences of SEQ ID NO: 1-119.
